(19) United States
(12) Patent Application Publication
(10) Pub. No.: US 2011/0009612 A1
(43) Pub. Date: Jan. 13, 2011

(54) AUTOMATED SOLUTION-PHASE ITERATIVE SYNTHESIS

(75) Inventors: NICOLA LUCIA POHL, AMES, IA (US); GISUN PARK, AMES, IA (US)

Correspondence Address: MCKEE, VOORHEES & SEASE, P.L.C., 801 GRAND AVENUE, SUITE 3200, DES MOINES, IA, 50309-2721, US

(73) Assignee: IOWA STATE UNIVERSITY RESEARCH FOUNDATION, INC., AMES, IA (US)

(21) Appl. No.: 12/886,644

(22) Filed: Sep. 21, 2010

Related U.S. Application Data
(62) Division of application No. 11/767,098, filed on Jun. 22, 2007.

(51) Int. Cl. C08B 37/00; C07H 1/00

(52) U.S. Cl. 536/123.1

(57) ABSTRACT

The first method for iterative solution-phase biomolecule synthesis is described. The method requires only 3 or fewer equivalents of building block at each coupling cycle, and incorporates a FSPE step at the end of each coupling/deprotection sequence to eliminate most byproducts.

### CROSS-REFERENCE TO RELATED APPLICATIONS

This is a divisional application of U.S. application Ser. No. 11/767,098 filed Jun. 22, 2007 which is hereby incorporated by reference in its entirety.

### FIELD OF THE INVENTION

This invention relates to an automated method of synthesizing oligosaccharides using fluorous tagging and automated fluorous solid phase extraction.

### BACKGROUND OF THE INVENTION

Polynucleotides, peptides/proteins, and carbohydrates are the three most important biomolecules in living organisms. Carbohydrates interact specifically with proteins to mediate biological processes that include inflammatory responses, pathogen invasion, cell differentiation, cell-cell communication, cell adhesion and development, and tumor cell metastasis. Information about these interactions would help illuminate the role of carbohydrates in the life cycles of organisms as well as foster the development of sugar-based therapeutics such as vaccines that intervene in these carbohydrate-protein interactions. Unfortunately, the molecular basis for many of these sugar-protein interactions is not understood, in part because homogeneous, well-defined carbohydrates are extremely difficult to obtain.

The only currently known method to do iterative organic chemistry (i.e. a sequence of organic reactions with purification steps in between each or most reaction steps) by a machine is by solid-phase chemistry. The current commercial methods to make nucleic acids and peptides are based on automated solid-phase methods (see e.g. U.S. Pat. No. 7,160,517, the disclosure of which is hereby incorporated by reference). However, for carbohydrates, no commercially available automated platform has yet emerged for oligosaccharide synthesis.

Biphasic (such as solid/liquid) reactions are inherently slower and less efficient than reactions carried out in which all the participants are in solution. To get around this problem, solid-phase processes require excesses of building blocks and reagents with up to 10-20 equivalents of a building block for each coupling cycle. When using 10 equivalents per coupling cycle, a hexasaccharide with quantitative coupling yields would translate into 6 equivalents in the final product and 54 equivalents in the waste bin.

Sugar building blocks require more steps to make than standard amino acid and nucleic acid building blocks. In this regard, synthesis of an oligosaccharide building block with appropriately masked functional groups can often require up to fourteen steps. Monitoring reaction progress is also much more difficult on a solid-phase than in solution as analytical methods are limited. Finally, solid-phase methods work best when all reaction steps progress in near quantitative yields. Compared to forming amide bonds in making peptides and phosphorous/oxygen bonds in making nucleic acids, quantitative yields in making glycosyl bonds are essentially unheard of. Reaction mistakes then build up on the solid-phase and cannot be removed until the end of the cycle. Dozens of automation platforms are designed to carry out a single reaction step at a time.

There has recently been increased interest in automated synthesis of oligosaccharides. For example, it is often of interest in examining structure-function relations involving sugars to generate a mixture of oligosaccharides having different residues at a particular position or varying in anomeric configuration at a glycosidic linkage. Furthermore, oligosaccharides having a desired activity, such as a high binding affinity to a given receptor or antibody, may be identified by generating a large number of random-sequence oligosaccharides, and screening these oligosaccharides to identify one or more having the desired binding affinity.

Accordingly, it is a primary objective of the present invention to provide an improved, automated method of synthesizing small molecules.

It is a further objective of the present invention to provide an automated method of synthesizing oligosaccharides.

It is a further objective of the present invention to provide an automated method of synthesizing oligosaccharides using fluorous tags under solution phase conditions.

It is a further objective of the present invention to provide an automated method of synthesizing oligosaccharides having reduced waste of building blocks.

It is still a further objective of the present invention to provide an automated method of synthesizing oligosaccharides having great flexibility in chemistry.

It is yet a further objective of the present invention to provide an automated method of synthesizing oligosaccharides having a direct interface with a microarray platform for compounds screening.

It is a further objective of the present invention to provide an automated method of synthesizing oligosaccharides that is accurate and relatively fast in comparison to non-automated methods.

The method and means of accomplishing each of the above objectives as well as others will become apparent from the detailed description of the invention which follows hereafter.

### SUMMARY OF THE INVENTION

The present invention describes the first automated iterative method to carry out solution phase chemistry as a means of synthesizing carbohydrate oligomers, and other small molecules including, but not limited to, glycosylated peptides, lipids, and polyketides. Current commercial methods for iterative synthesis rely primarily on solid-phase methods. The method and device involves a reactor which contains a fluorous-tag on which a component of the product to be synthesized is coupled to a platform for fluorous solid-phase extraction to remove reaction by-products. One embodiment of the invention relates to an apparatus for the efficient synthesis of oligosaccharides on a fluorous tag, i.e. formed by subunit addition to terminal subunits covalently attached to a fluorous tag. The progress of coupling and deprotection reactions which occur in the synthesis of oligosaccharides is monitored by standard solution-phase methods. The solution-phase approach to oligosaccharide synthesis requires only 1-3 equivalents of glycosyl donor to be used in each coupling cycle, rather than the larger excesses required in solid-phase methods.

### DETAILED DESCRIPTION OF THE DRAWINGS

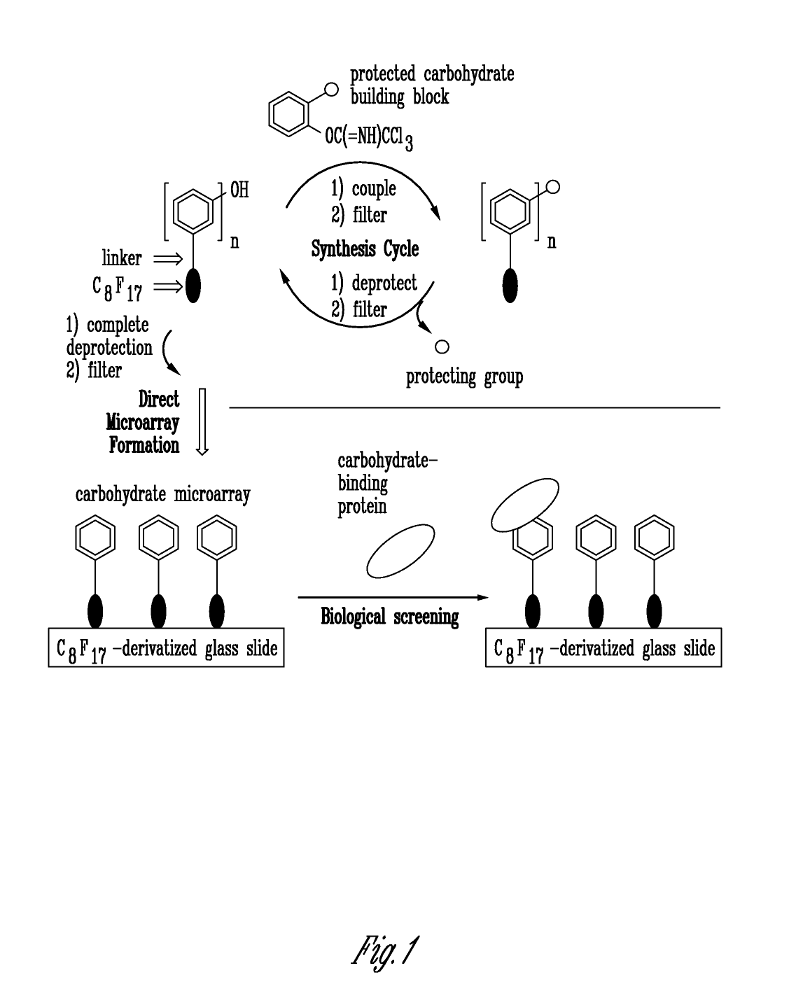

FIG. 1 illustrates a basic oligosaccharide synthesis scheme.

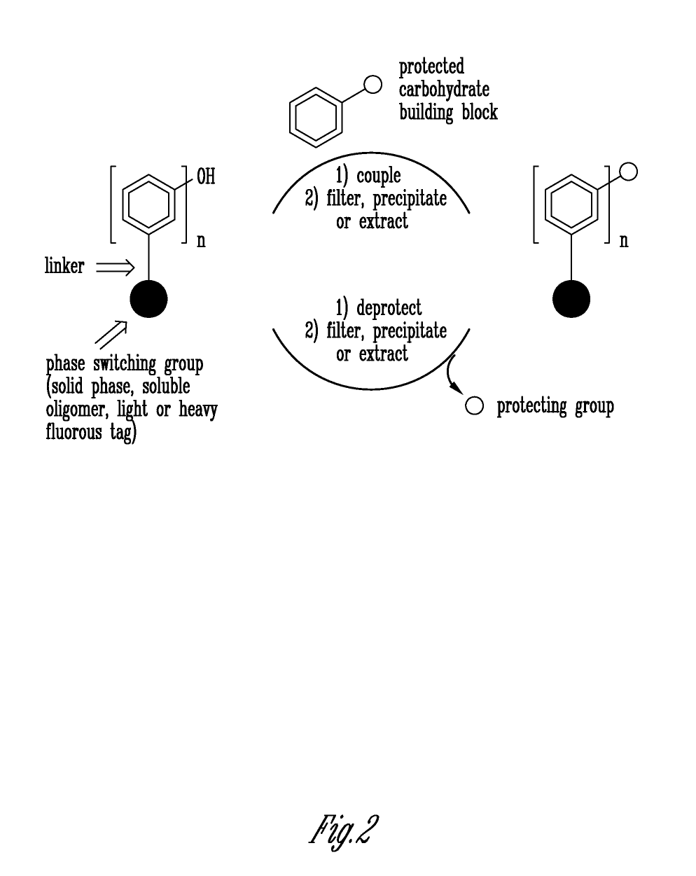

FIG. 2 illustrates a basic scheme for phase-switching approaches to iterative carbohydrate synthesis.

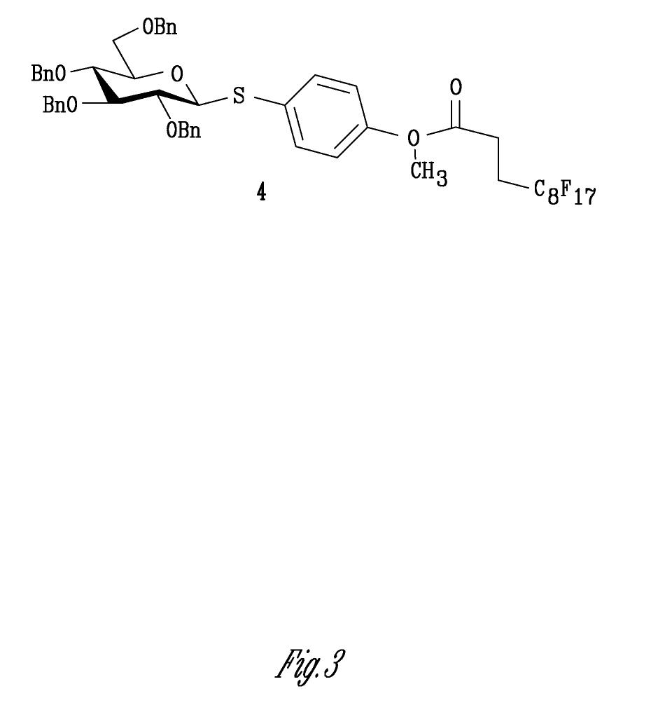

FIG. 3 illustrates a single fluorous tag for a recyclable activating group for glycosylations.

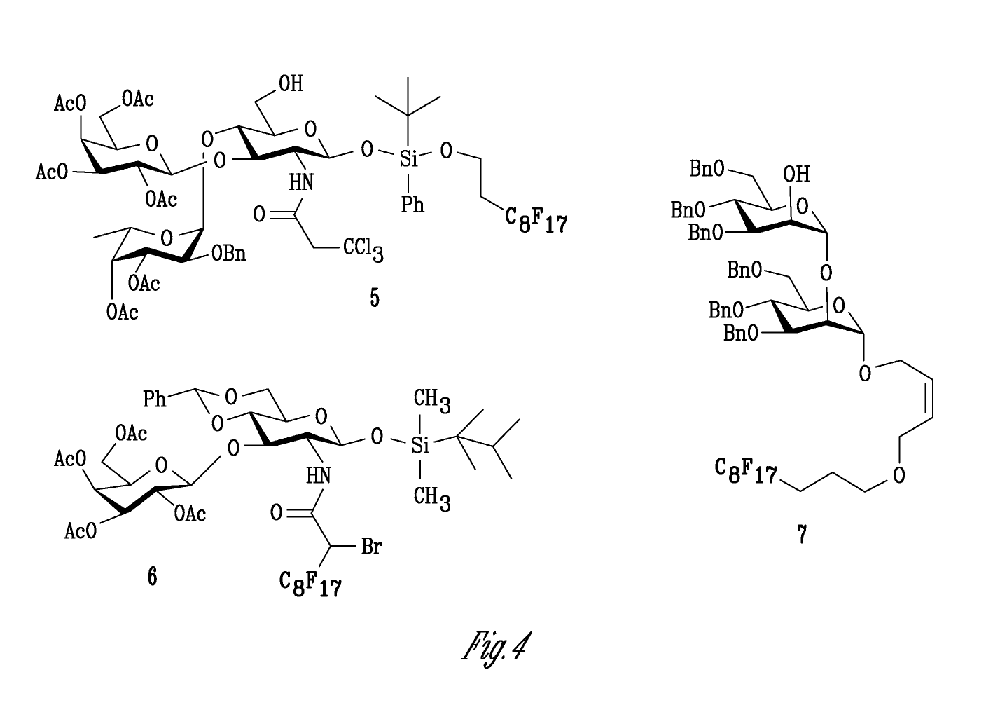

FIG. 4 illustrates protecting groups with single fluorous tags for the synthesis of carbohydrates with fluorous solid-phase extraction of intermediates.

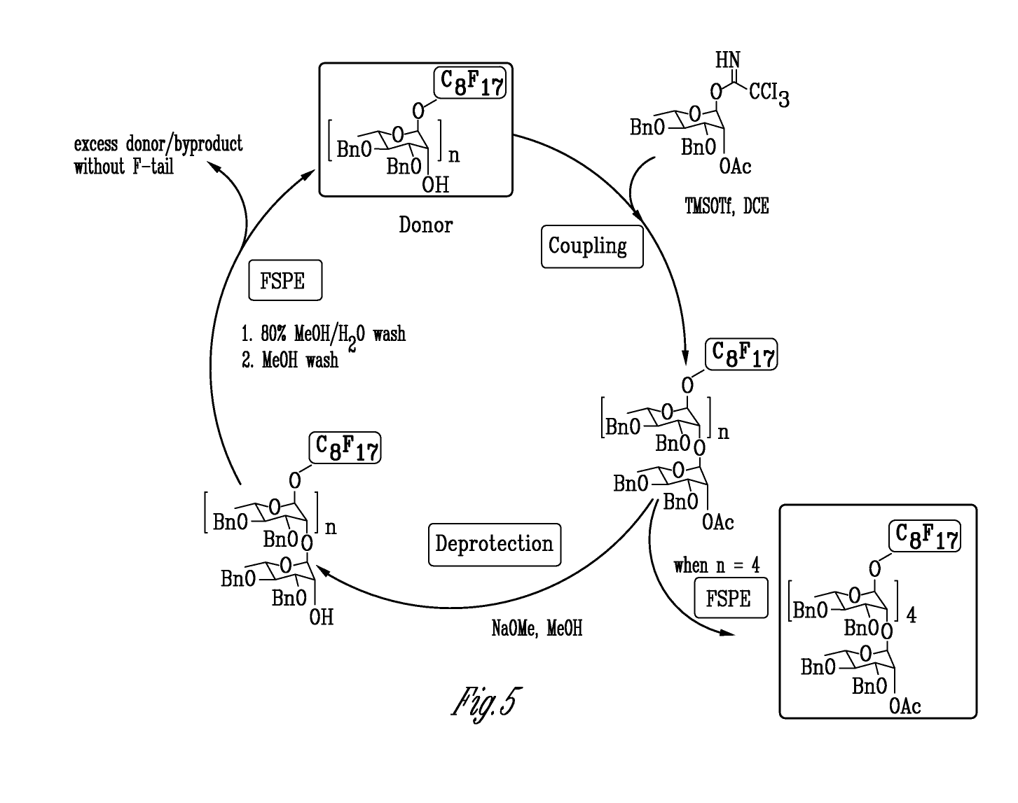

FIG. 5 illustrates a preferred automation protocol for synthesizing polyrhamnose as described in Example 1.

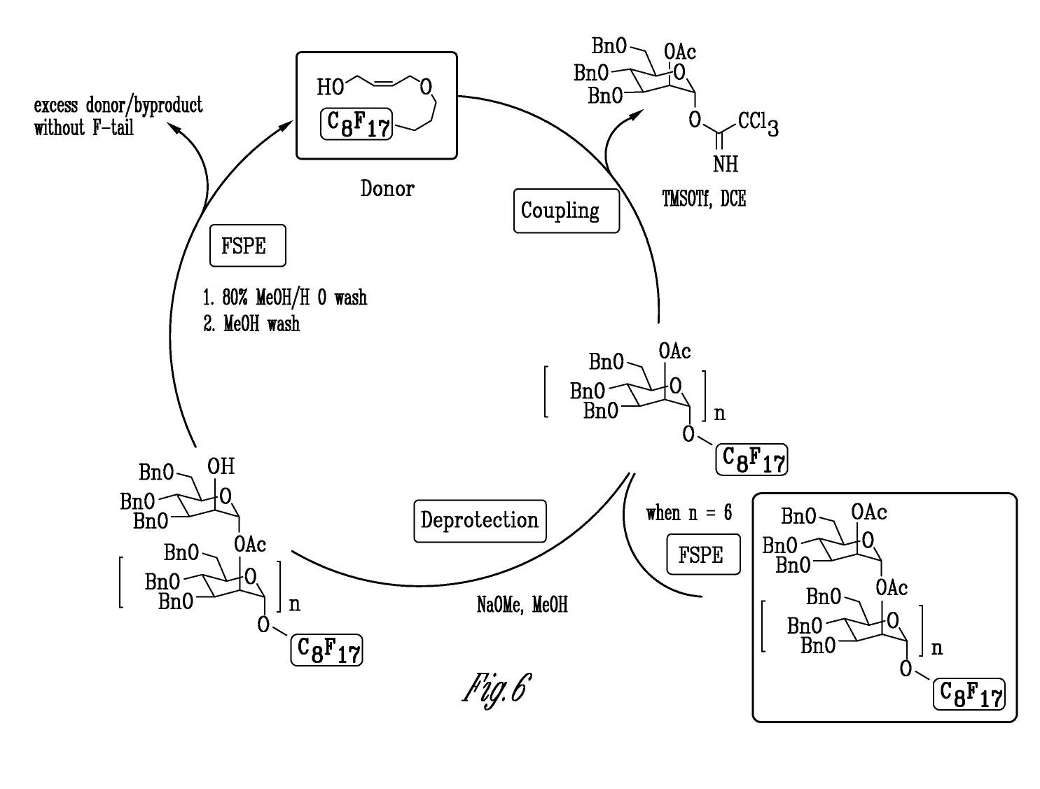

FIG. 6 illustrates a preferred automation protocol for synthesizing polymannose as described in Example 1.

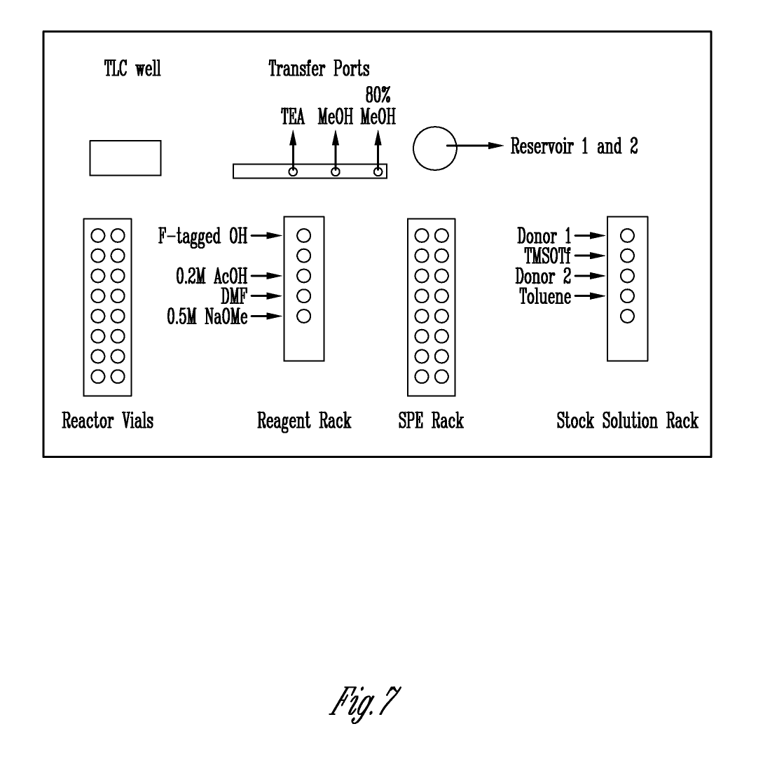

FIG. 7 illustrates an embodiment of an automation platform using the ASW 1000 platform using the methods of the invention.

### DETAILED DESCRIPTION OF THE PREFERRED EMBODIMENT

The present invention relates to the development of an automated method for synthesizing oligosaccharides using solution phase conditions. The oligosaccharide product is of a type which includes a linear sequence of four or more glycosyl units linked to one another by glycosidic linkages. The sequence starts with a first glycosyl unit at a nonreducing end, concludes with a final glycosyl unit at a reducing end, and includes two or more intermediate glycosyl units sequentially arrayed between the first and final glycosyl units. The process is of a type which includes a condensation of protected glycosyl donors or protected glycosyl donor/acceptors with protected glycosyl donor/acceptors or protected glycosyl acceptors for producing a protected oligosaccharide intermediate. The protected oligosaccharide intermediate is then deprotected for producing the oligosaccharide product.

More particularly, the improvement is directed towards an automation process for iterative solution-phase biomolecule synthesis whereby 2 or fewer equivalents of building block are needed at each coupling cycle. The method also employs a fluorous solid phase extraction (FSPE) step at the end of each coupling/deprotection sequence to eliminate most byproducts. FIG. 1 generally illustrates the general oligosaccharide synthesis process.

The structure of oligosaccharides consists of many glycosidic linkages, which often can be controlled stereospecifically with the help of a neighboring group participant. The chemistry of the glycosylation reactions requires an activated donor building block in addition to a free hydroxyl group which acts as the nucleophile on an acceptor building block.

Iterative biopolymer synthesis is often facilitated by the use of soluble or solid-phase supports to simplify the purification of intermediates. (FIG. 2). For example, solid-phase carbohydrate synthesis allows excess reagents required for reaction completion to be washed off between steps in a process that has been automated. Plante, O. J. et al., Science, 2001, 291, 1523-1527. Alternatively, to avoid poor reaction kinetics inherent to biphasic systems, soluble tags with unique physical properties can be attached to the growing chain to aid in purification of intermediates by tag precipitation, extraction into a liquid phase, or affinity chromatography/solid-phase extraction. Ito, Y. et al., Chem. Eur. J. 2002, 8, 3077-3084 and references therein. Because tag precipitation is not quantitative, tags for extraction methods are attractive options.

Soluble fluorocarbon tags have been employed in the synthesis of a variety of carbohydrates. Fluorocarbons will phase separate from aqueous or conventional organic solvents. Several fluorocarbon chains can be incorporated into a protecting group to allow extraction of the compound containing the “heavy” fluorous tag into a liquid fluorocarbon layer or a single fluorocarbon chain, a “light” fluorous tag, can capture the tagged molecule by fluorous-derivatized silica gel in a solid-phase extraction process. Horvath, I. T. Acc. Chem. Res. 1998, 31, 641-650. Both “heavy” and “light” fluorous tags have been developed specifically for the challenges of oligosaccharide synthesis.

Nonenzymatic carbohydrate synthesis usually relies on protecting groups to permanently mask some hydroxyl and other functional groups and to temporarily block future reaction sites. These protecting groups are perfect locations for the introduction of fluorocarbon tags that allow liquid-liquid extraction of the reaction product away from excess reagents. The first protecting group introduced for this purpose to aid sugar synthesis was a variation of the commonly-used benzyl group. Three fluorocarbon chains attached to a silicon modify a benzyl group and allow extraction of molecules protected with the group into perfluorohexanes. Curran, D. P., et al., Tetrahedron Lett. 1998, 39, 4937-4940. Unfortunately, the necessity to include multiple fluorocarbon tails for efficient liquid-liquid extraction also limits the solubility of the compounds in the nonfluorocarbon solvents required for a range of reaction types. Additionally, the large protecting groups can complicate spectral interpretation for characterization and a substantial amount of the molecular weight of the intermediates is accounted for by the fluorocarbon tags. Nonetheless, the tags allow iterative carbohydrate synthesis with minimal chromatography and with the benefits of solution phase reaction kinetics and reaction monitoring not possible by solid-phase approaches.

The addition of only one fluorocarbon chain to a protected carbohydrate renders the molecule separable from non-tagged compounds not by liquid-liquid extraction but by solid-phase extraction (SPE) instead. The reaction mixture is loaded on fluorous silica gel, untagged compounds are eluted, and then a change of solvent allows elution of the pure tagged compound. Curran, D. P. et al. Separations with Fluorous Silica Gel and Related Materials. In The Handbook of Fluorous Chemistry, Gladysz, J.; Horvath, I.; Curran, D. P.; Wiley-VCH: Weinheim, 2004; pp. 101-127. Several carbohydrate protecting groups as well as an anomeric activating group for glycosylation reactions have been designed with single fluorous tags to simplify purification schemes.

Addition of a fluorous tag to a thiol anomeric activating group creates a glycosylation building block (FIG. 3) that can easily be purified by SPE. The thiol byproduct after glycosylation can be readily removed by SPE and recycled after reduction of any disulfide formed. In addition to the benefits of purification ease, the fluorous tag also renders the thiol less repugnant.

Fluorous protecting groups have also been used to facilitate iterative carbohydrate synthesis by solid-phase extraction of growing chain intermediates. A fluorous version of a silicon protecting group was used to protect the anomeric position of a glucosamine building block and build up the Lewis a trisaccharide 5 (FIG. 4) with intermediates purified by fluorous SPE. Manzoni, L. Chem. Commun. 2003, 2930-2931. A related fluorous silyl group has been used not in iterative synthesis but to cap oligosaccharides made on solid-phase for isolation of tagged sequences by SPE. Palmacci, E. E. et al., Angew. Chem. Int. Ed. 2001, 40, 4433-4437. More recently, a fluorous version of a carbamate nitrogen protecting group was developed and applied to the synthesis of a disaccharide 6. (FIG. 4). Manzoni, L. et al., Org. Lett. 2006, 8, 955-957. The group can be synthesized in three steps and removed with exchange to an acetyl group using zinc in acetic anhydride with triethylamine. Finally, a fluorous version of the allyl protecting group has also been developed for facile purification of intermediates in the synthesis of polymannosides such as 7, for example. (FIG. 4). The fluorous allyl group allows fluorous SPE purification of intermediates and can be removed using standard palladium-mediated deallylation conditions.

Unlike solid-phase approaches, iterative fluorous-phase synthesis of any molecule class has never been automated. Automation is key to gaining the benefits of facile library synthesis seen with the automation of both peptide and nucleic acid synthesis. As noted, chemistry amenable to automation for iterative oligosaccharide synthesis based on fluorous tags has been developed, but demonstration of its automation has remained to be seen, until development of the methods of the present invention. This strategy can be readily used with a range of chemistries and thereby promises broad utility and applicability even beyond glycomics.

Fluorous carbohydrate synthesis has several important advantages over other methods including: 1) the use of only 1.5 to 3 eq of each building block rather than a large excess since the reactions are solution phase rather than solid phase; and 2) allowing for a simple purification and compound identification after each coupling cycle, which is not possible with solid phase methods.

In manufacturing the oligosaccharides in accordance with the present invention, as already noted, appropriate monomers are those having a free hydroxyl group. Examples of such monomers include, but are not limited to, carbohydrates that may be glycosides, aminoglycosides, or ether- or amino-linked sugars, where the coupling takes place through a non-glycosidic position. The building block mono- or oligosaccharide-donors may be any activated sugar including, but not limited to, orthoesters, thioorthoesters, cyanoalkylidene derivatives, 1-O-acyl sugars, amino sugars, acetimidates, trichloroacetimidates, thioglycosides, aminoglycosides, amino-oligosaccharides, glycosylamines of oligosaccharides, glycosyl thiocyanates, pentenyl glycosides, pentenoylglycosides, isoprenyl glycosides, glycals, tetramethylphosphoro diamidates, sugar diazirines, selenoglycosides, phosphorodithioates, glycosyl-dialkylphosphites, glycosylsulphoxides and glycosylfluorides. The individual saccharide residues are attached directly to linkers via their anomeric carbons, and the linkers have the characteristic that at least one set of conditions for releasing the saccharides, oligosaccharides, and/or polysaccharides from the solid support provides saccharides, oligosaccharides, and/or polysaccharides wherein the residues that were attached directly to the solid support are transformed into glycosyl donors. In one embodiment of this invention, the molecule is mannose, which may be used in the synthesis of polymannoses, such as dimmanopyranoside.

The monomer unit of the present invention can be immobilized on a variety of solid or soluble supports including, but not limited to, polystyrene, polyethylene, Teflon, silicon gel beads, hydrophobized silica, mica, filter paper (e.g. nylon, cellulose, and nitrocellulose), glass beads and slides, gold and all separation media such as silica gel, sephadex, and other chromatographic media. These and other such supports are well known in the art.

As noted, the connection of the monomer to the solid support is accomplished through a linker which can be viewed as a support-bound protecting group. A variety of linkers have previously been prepared for the attachment of hydroxyl groups to the solid phase. Linkers for solid and solution support synthesis are well known in the art and include, but are not limited to, silyl ethers, thioethers, succinyl esters and nitrobenzyl ethers. Such linkers are well known in the art. A preferred linker for use in this invention is an alkene or thiol linker.

One or more of the molecules to be coupled is tagged with a soluble fluorous tag, which allows adsorption of the molecule to a solid support for purification. such as, for example, a fluorous-tagged molecule. The procedure for tagging molecules having protecting groups is well known in the art. The conditions will vary depending upon the tag(s) chosen, substrate used, etc. An exemplary and well known reference in this respect is T. W. Green, P. G. M. Wuts, Protective Groups in Organic Synthesis, Wiley-Interscience, N.Y., 1999, the contents of which are specifically incorporated herein by reference. This book provides detailed information to persons skilled in the art regarding the tagging conditions/procedures to use depending on the protecting group selected. Once the monomer, oligomer or polymer is tethered to the solid support, the molecule is deprotected, thereby liberating the hydroxyl protecting group. The molecule may be deprotected while still on the solid support, or deprotected following its elution back into the reaction solution.

The synthesis of oligosaccharides, oligonucleotides, etc. on the solid or soluble support requires the development of a coupling cycle which consists of a series of operations required to elongate the growing chain by one unit. Attachment of an appropriately protected monomer through its reducing end is followed by removal of the hydroxyl protecting group from a uniquely designated hydroxyl group. Washing steps to clean the support follow.

The exposed hydroxyl group functions as a glycosyl acceptor during the coupling step by reaction with a glycosyl donor in the presence of an acid, such as trimethylsilyltriflate or boron trifluoride etherate as an activator. After several washing steps any unreacted glycosyl acceptor hydroxyl groups are optionally capped off by reaction with acetic anhydride to prevent the formation of deletion sequences by reaction of these sites during subsequent coupling cycles. Repetition of this cycle leads to the formation of compounds containing β-glycosidic or α-glycosidic linkages. Cleavage from the solid support and final deprotection followed by purification then yield the desired oligosaccharide product.

In automating the above-referenced methods, the inventors reengineered an existing robotic driven automated synthesis workstation that includes parallel reaction devices for synthesizing and screening combinatorial libraries. These reactors synthesize milligram to gram quantities of materials, which can be screened or analyzed by various techniques including gas chromatography, FT-IR, and UV-Visible spectroscopy. In this regard, the ASW 1000 manufactured by Chemspeed was altered for this purpose. FIG. 7 illustrates one possible set-up for the automated platform of this invention using the ASW 1000. However, it would be readily appreciated to persons skilled in the art that other brands/types of automated synthesis workstations could also be appropriately altered to perform the methods in accordance with the teachings of the invention, i.e. Endeavor, Neptune, FlexChem, Reacto-Stations, etc. Furthermore, persons skilled in the art would also appreciate that platforms to perform the methods described herein can be independently constructed in lieu of modifying an existing automated platform.

In addition to parallel reaction devices, a system of the invention typically includes other vessels and/or reagents to and from the reaction blocks or other vessels. Additional details regarding solid support containers that are optionally used in the devices of the present invention, including those that provide for molecular tracking and identification are described in, e.g., U.S. Pat. No. 6,136,274 to Nova et al., issued Oct. 24, 2000, which is incorporated by reference in its entirety for all purposes. Other system components optionally include, e.g., vacuum manifold systems for eluting fluidic materials from reaction wells, incubators/ovens for regulating temperatures within reaction wells, centrifuges, shakers or other agitation devices, or the like. The systems of the invention also typically include a detection system (e.g., a mass spectrometer or the like) to detect chemical or physical properties of selected members of, e.g., synthesized libraries, and a computer (e.g., an information appliance, digital device, or the like) operably connected to the handling, detection, and/or other systems.

Additional details relating to synthesis systems, which are optionally adapted for use with the devices of the invention, and to the automation of combinatorial synthetic methods are described in, e.g., Cargill and Maiefski (1996) “Automated combinatorial chemistry on solid phase,” Lab. Robotics. Automation 8: 139-148, Zuckermann et al. (1992) “Design, construction and application of a fully automated equimolar peptide mixture synthesizer,” Int. J. Peptide Prot. Res. 40: 497-506, Castelino et al. (2000) “Automated sample storage for drug discovery,” Chim. Oggi. 17: 32-35, Davis and Swayze (2000) “Automated solid-phase synthesis of linear nitrogen-linked compounds,” Biotechnol. Bioeng. 71: 19-27, Groger et al. (2000) “1,3,5-Triazines, versatile industrial building blocks: Synthetic approaches and applications,” Chim. Oggi. 18: 12-16, Haag (2000) “Chemspeed Ltd.: Automated and unattended parallel synthesis integrating work-up and analysis,” Chimia 54: 163-164, Hu et al. (2000) “Automated solid-phase synthesis and photophysical properties of oligodeoxynucleotides labeled at 5′-aminothymidine with Ru(bpy)(2)(4-m-4′-cam-bpy)(2+),” Inorg. Chem. 39: 2500-2504, Lewis et al. (2000) “Automated high-throughput quantification of combinatorial arrays,” American Pharmaceutical Review 3: 63-68, North (2000) “Implementation of analytical technologies in a pharmaceutical development organization-looking into the next millennium,” Journal of Automated Methods and Management in Chemistry 22: 41-45, and Keifer et al. (2000) “Direct-injection NMR (DI-NMR): A flow NMR technique for the analysis of combinatorial chemistry libraries,” Journal of Combinatorial Chemistry 2; 151-171.

The handling systems of the invention typically incorporate one or more controllers, either as separate or integral components, which are generally utilized, e.g., to regulate the quantities of reagents dispensed. A variety of available robotic elements (robotic arms, movable platforms, etc.) can be used or modified for these purposes.

To illustrate, controllers typically direct dipping of handling elements of the handling systems into, e.g., selected reaction wells of reaction blocks, wells on micro-well plates, or other reaction vessels, to dispense or extract, e.g., selected beads or other solid supports. Typically, the controller systems of the present invention are appropriately configured to receive or interface with a parallel reaction device or other system component. For example, the controller optionally includes a stage upon which the reaction devices of the invention are disposed or mounted to facilitate appropriate interfacing among, e.g., a bead/fluid handler and/or detector and a particular parallel reaction device. Typically, the stage includes an appropriate mounting/alignment structural element, such as alignment pins and/or holes, a nesting well, or the like, e.g., to facilitate proper device alignment.

The systems of the present invention optionally include various signal detectors, e.g., which detect mass, concentration, fluorescence, phosphorescence, radioactivity, pH, charge, absorbance, refractive index, luminescence, temperature, magnetism, or the like. Detectors optionally monitor one or a plurality of signals from upstream and/or downstream of the performance of, e.g., a given synthesis step. For example, the detector optionally monitors a plurality of optical signals, which correspond in position to “real time” results. Example detectors or sensors include photomultiplier tubes, CCD arrays, optical sensors, temperature sensors, pressure sensors, pH sensors, conductivity sensors, scanning detectors, or the like. The detector optionally moves relative to assay components, or alternatively, assay components, such as samples of selected synthesis products move relative to the detector. Optionally, the systems of the present invention include multiple detectors. Each of these types of sensors is optionally readily incorporated into the systems described herein. In these systems, such detectors are typically placed either in or adjacent to, e.g., a particular reaction vessel, such that the detector is within sensory communication with the reaction vessel. The detector optionally includes or is operably linked to a computer, e.g., which has system software for converting detector signal information into assay result information or the like.

The detector optionally exists as a separate unit, or is integrated with the handling or controller system, into a single instrument. Integration of these functions into a single unit facilitates connection of these instruments with the computer (described below), by permitting the use of few or a single communication port(s) for transmitting information between system components.

Specific detection systems that are optionally used in the present invention include, e.g., a mass spectrometer, an emission spectroscope, a fluorescence spectroscope, a phosphorescence spectroscope, a luminescence spectroscope, a spectrophotometer, a photometer, a nuclear magnetic resonance spectrometer, an electron paramagnetic resonance spectrometer, an electron spin resonance spectroscope, a turbidimeter, a nephelometer, a Raman spectroscope, a refractometer, an interferometer, an x-ray diffraction analyzer, an electron diffraction analyzer, a polarimeter, an optical rotary dispersion analyzer, a circular dichroism spectrometer, a potentiometer, a chronopotentiometer, a coulometer, an amperometer, a conductometer, a gravimeter, a thermal gravimeter, a titrimeter, a differential scanning colorimeter, a radioactive activation analyzer, a radioactive isotopic dilution analyzer, or the like.

As noted above, the systems of the present invention optionally include a computer (or other information appliance) operably connected to or included within various system components. The computer typically includes system software that directs the handling and detection systems to, e.g., segregate or distribute solid supports into selected reaction wells or other vessels, deliver various reagents (e.g., different components or building blocks, scaffolds, or the like) to selected reaction wells of reaction blocks, deliver gases to maintain inert environments within reaction wells via syringe needles, or the like. Additionally, the handling/controller system and/or the detection system is/are optionally coupled to an appropriately programmed processor or computer which functions to instruct the operation of these instruments in accordance with preprogrammed or user input instructions, receive data and information from these instruments, and interpret, manipulate and report this information to the user. As such, the computer is typically appropriately coupled to one or both of these instruments (e.g., including an analog to digital or digital to analog converter as needed).

Standard desktop applications such as word processing software (e.g., Microsoft Word™ or Corel WordPerfect™) and database software (e.g., spreadsheet software such as Microsoft Excel™, Corel Quattro Pro™, or database programs such as Microsoft Access™ or Paradox™) can be adapted to the present invention by inputting character strings corresponding to reagents or masses thereof. For example, the systems optionally include the foregoing software having the appropriate reagent information, e.g., used in conjunction with a user interface (e.g., a GUI in a standard operating system such as a Windows, Macintosh or LINUX system) to manipulate reagent information.

The computer can be, e.g., a PC (Intel x86 or Pentium chip-compatible DOS™, OS2™, WINDOWS™, WINDOWS NT™, WINDOWS95™, WINDOWS98™, LINUX-based machine, a MACINTOSH™, Power PC, or a UNIX-based (e.g., SUN™ work station) machine) or other common commercially available computer which is known to one of skill Software for performing, e.g., library synthesis is optionally easily constructed by one of skill using a standard programming language such as Visual basic, Fortran, Basic, Java, or the like. Any controller or computer optionally includes a monitor which is often a cathode ray tube (“CRT”) display, a flat panel display (e.g., active matrix liquid crystal display, liquid crystal display), or others. Computer circuitry is often placed in a box, which includes numerous integrated circuit chips, such as a microprocessor, memory, interface circuits, and others. The box also optionally includes a hard disk drive, a floppy disk drive, a high capacity removable drive such as a writeable CD-ROM, and other common peripheral elements. Inputting devices such as a keyboard or mouse optionally provide for input from a user.

The computer typically includes appropriate software for receiving user instructions, either in the form of user input into a set of parameter fields, e.g., in a GUI, or in the form of preprogrammed instructions, e.g., preprogrammed for a variety of different specific operations. The software then converts these instructions to appropriate language for instructing the operation of one or more controllers to carry out the desired operation, e.g., varying or selecting the rate or mode of movement of various system components, directing X-Y-Z translation of the bead/fluid or other reagent handler, or of one or more micro-well plates or other reaction vessels, or the like. The computer then receives the data from the one or more sensors/detectors included within the system, and interprets the data, either provides it in a user understood format, or uses that data to initiate further controller instructions, in accordance with the programming, e.g., such as in monitoring reaction temperatures, regulating agitation rates, or the like.

The reaction devices of the present invention are designed primarily for use in essentially solution-phase organic synthesis. The devices of the invention provide particular utility where numerous, individual reactions are performed simultaneously and, e.g., where filtration is a necessary step during the synthesis and/or workup process. Other exemplary uses for the parallel reaction devices, or device components, of the invention include performing multiple, simultaneous chromatographic or affinity-based separations/purifications. To illustrate, each reaction well of a device optionally serves as a column for chromatographic separation of chemical mixtures on, e.g., silica gel, alumina, or many other adsorbents/resins that are commonly known in the relevant art. The elution of samples or other materials is typically gravity-based or dependent on an applied pressure. Additional details regarding synthetic pathways, separations, and other processes optionally performed in the devices of the invention are described in, e.g., Seneci, Solid-Phase Synthesis and Combinatorial Technologies, John Wiley & Sons, Inc. (2000), Albericio and Kates, Solid-Phase Synthesis: A Practical Guide, Marcel Dekker (2000), An and Cook (2000) “Methodologies for generating solution-phase combinatorial libraries,” Chem. Rev. 100: 3311-3340, Wu (Ed), Column Handbook for Size Exclusion Chromatography, Harcourt Brace & Company (1998), and in the references cited therein. Other general resources include, e.g., March, Advanced Organic Chemistry: Reactions, Mechanisms, and Structure, 4.sup.th Ed., John Wiley & Sons, Inc. (1992), Smith and March, March's Advanced Organic Chemistry: Reactions, Mechanisms, and Structure, 5.sup.th Ed., John Wiley & Sons, Inc. (2001), Carey and Sundberg, Advanced Organic Chemistry Part A: Structure and Mechanism, 4.sup.th Ed., Plenum Press (2000), and in the references provided therein. The present invention also provides kits that include parallel reaction devices, or components of such devices.

The preferred device for use in the present invention, namely the Chemspeed ASW 1000, is a fully automated system for unattended parallel synthesis, reagent preparation, product analysis and purification, and can accommodate reaction blocks to run up to 80 reactions in parallel with an option to go up to 112 runs/cycle and can perform liquid-liquid and solid-phase extractions. This instrument has temperature control, can perform eight reactions in parallel for library synthesis, and can automatically load fluorous solid-phase extraction columns. The ASW 1000 workstation is based on a Gilson XL233 sample processor platform which can hold up to six syringes for liquid handling. The ASW can deliver reagents while shaking (up to 1,400 rpms), heating and cooling (−70° C. to 150° C.). This workstation also allows the following on-line processes: solvent evaporation, filtration, TLC to spot up to 32 reactions, and Rheodyne valves to interface with HPLC or HPLC/MS systems. The workstation also has an output to 96 deep-well plates.

The inventors made several changes to the commercially-available synthesizer in order to improve the performance of the various steps and functions of the present invention, as set forth in more detail below. The machine was equipped with several reagent bottles, some for washing and elution of the solid-phase extraction resin, and others for glycosylation and deprotection reagents. A cycle was programmed to operate all steps without operator intervention, so that the machine controlled the delivery of all reagents and donor species to the reaction vessel, as well as mixing of the contents of the vessel. The plate to hold solid-phase extraction cartridges was machined to hold the larger cartridges required for solid-phase extraction using fluorous silica gel rather than the standard common silica gel.

The automated oligosaccharide synthesizer of the present invention generally consists of a reaction vessel, two or more donor vessels containing monosaccharide donor solutions, an activator vessel containing monosaccharide donor solutions, an activator vessel containing an activating reagent solution, a deblocking vessel containing a deblocking reagent solution, a blocking vessel containing a blocking reagent solution, three solvent vessels containing solvent solutions, a solution transfer system, a temperature control unit for regulating the temperature of the reaction vessel (i.e., maintaining the temperature of the reaction vessel at a desired temperature(s)), and a computer which can be preprogrammed to automatically control the solution transfer system, the evaporation unit, and the temperature control unit.

The donor vessels can hold any suitable glycosyl donor solution, such as a glycosyl trichloroacetimidate or a glycosyl phosphate. The activator vessel can hold any suitable activating reagent solution. Generally, Lewis acids have shown to be conductive to the formation, i.e., synthesis, of oligosaccharides and therefore can be utilized as an appropriate activator. Thus, the activator vessel can contain a solution comprising a silyl trifluoromethanesulfonate or, alternatively, can contain a solution comprising trimethylsilyl trifluoromethanesulfonate. The deblocking vessel can hold any suitable deblocking reagent solution, such as a solution containing sodium methoxide or hydrazine. The blocking vessel can contain any suitable blocking reagent solution, such as a solution containing benzyl trichloroacetimidate or a carboxylic acid. One such suitable carboxylic acid is levulinic acid. The plurality of solvent vessels can contain any suitable solvents solutions, such as dichloromethane, THF, and methanol, amongst others.

The solution transfer system must be capable of transferring the donor, activating, deblocking, blocking and solvent solutions from their respective vessels to the reaction vessel. Due to the moisture sensitivity of certain glycosylation reactions, the solution transfer system should also be capable of maintaining the reagents of the apparatus under an inert gas atmosphere, i.e., maintain the reagents under positive pressure. The solution transfer system described in U.S. Pat. Nos. 5,186,898 and 7,160,517 (the disclosures of which are hereby specifically incorporated by reference) are also system which would be suitable for the synthesis of oligosaccharides in accordance with the present invention.

As already noted, the computer can be any suitable computing device for controlling the operations of the solution transfer system, the evaporation unit, and the temperature control unit, such as a personal computer or workstation, for example. The computer can be preprogrammed so as to automatically control the operations of the solution transfer system; the coupling, washing, protecting/capping (i.e., blocking), and deprotecting cycles for a given protocol can be preprogrammed into the computer. In this way, the automated solution-phase synthesis of oligosaccharides can be controlled and achieved. Additionally, the computer can be a device which is separate from the solution transfer system, or the computer can be integral to the solution transfer system. If the computer and the solution transfer system are separate devices, then a suitable data communication path, such as a communication port/cable or IR data link, between the two devices must be present Likewise, if automatic temperature control of the reaction vessel is desired, then the computer can be preprogrammed with the desired temperature protocol so as to control the operations of the temperature control unit. For the automatic control of the temperature control unit via the computer, the computer must be in data communication with the temperature control unit.

The temperature control unit can be any suitable device which is capable of regulating and maintain the temperature of the reaction vessel at a desired temperature(s). Several of the external refrigerated circulators available from the Julabo USA, Inc., Allentown, Pa., can be used as an acceptable temperature control unit for example. To accomplish the automated solution-phase synthesis of many different types of oligosaccharides, the temperature control unit should be capable of maintaining the temperate of the reaction vessel at a set temperature of between −25° C. and 40° C., and preferably at a set temperature of between −80° C. and +60° C. The coolant of the temperature control unit can be circulated around the reaction vessel via a sleeve which can surround the reaction vessel and which is connected to the temperature control unit via input and output pathways. Alternatively, the reaction vessel can be a double-walled structure wherein the external cavity of the double-walled structure accommodates the coolant of the temperature control unit. The temperature of the reaction vessel can be established by pre-programming the temperature control unit to the desired temperature and then allowing the coolant to circulate around the reaction vessel for some pre-established “cold soak” period, such as five minutes, for example. Alternatively, the temperature control unit can have a temperature sensor placed on the wall of the reaction vessel so as to obtain real-time temperature measurements of the actual reaction vessel cavity, i.e., where the automated synthesis of the oligosaccharides are to take place. Thus, the temperature sensor can provide feedback data to the temperature control unit so that the actual temperature of the reaction vessel can more properly be maintained.

One embodiment of a double-walled cooled reaction vessel in accordance with the invention includes two cavities: a first cavity accommodates the synthesis of the oligosaccharides; the second cavity accommodates the coolant of the temperature control unit. The coolant of the temperature control unit circulates through the second cavity via conduits. These conduits may be comprised of any suitable materials, such as rubberized materials or metallic materials, for example. The conduits can be secured to the two opening found in the exterior surface of the double-walled reaction vessel via mechanical clamping, tapes, bonds, epoxies etc. The double-walled cooled reaction vessel can be made of glass or any other suitable material, such as titanium, for example.

The various solutions can be introduced into the cavity via the solution transfer system. These solutions, likewise, can be forced out of cavity (to be captured as waste) through operation of the solution transfer system by the introduction of additional solution or through the introduction of a compressed inert gas.

A first obstacle the inventors had to overcome was to equip the commercial machine to handle a larger amount of material. In this regard, the solid-phase extraction (SPE) cartridge rack as purchased with the machine only hold 1 mL cartridges, which are large enough to hold enough resin for a simple filtration through silica gel, but do not hold enough resin to do a fluorous solid-phase extraction for a 25-50 micromole scale synthesis, such as described herein. For this reason, the cartridge rack is preferably specially machined to cradle larger cartridges holding 0.5 grams or more of fluorous silica gel resin. The inventors also modified the robotic needle movement to perform the specific oligosaccharide synthesis steps, for example, to allow the needle to reach the bottom of the vial under the SPE rack due to the alteration that was made in order to use longer FSPE cartridges.

Another preferred modification to accommodate automated oligosaccharide synthesis was to provide a means for the needle to withdraw product sample from the vials completely. In this regard, the inventors substituted a flat-tipped needle for the beveled needle in the device in order to maximize liquid transfers, and prepierced septa were added to all reagent bottles and solid-phase extraction cartridges to accommodate this substitution. Since the automated platform is conventionally programmed to perform a series of parallel reactions rather than iterative cycle, such a modification would not have otherwise been contemplated by persons skilled in the art.

SPE further requires a certain amount of pressure in order to accomplish filtration, i.e. greater than ambient pressure, which typically requires further modifying an existing commercial synthesizer in order to increase the degree of pressure provided in this step. This may be accomplished using various means understood in the art, such as by placing caps on the cartridges to increase pressure and provide an air tight system. The amount of pressure required will also depend upon the size of the cartridge used in the system, i.e. increasing the size of the cartridge will increase the level of pressure. The pressure should be increased to a level so as to provide at least a consistent and acceptable rate of filtration in the system for the compound being synthesized. Persons skilled in the art would readily understand how this may be accomplished, as well as the level of pressure suitable for the intended purpose.

Automated synthesis with the commercial synthesizer also provided significant splashing of the sample around vial walls during evaporation cycles, causing buildup of unreacted material on the sides of the vial, thus significantly reducing yields. This problem may be solved, for example, by co-evaporation of the sample with a solvent that lowers the viscosity of the reaction mixture. Toluene is a preferred solvent for this purpose, in that it acts as an aziotrope with the primary solvent, thus causing the combination of solvents to boil at a lower temperature than either solvent would by itself. Other solvents that may be used for this purpose include, but are not limited to, xylene and benzene. A FSPE step is not usually necessary directly after the glycoslyation step and can be eliminated at this stage to save time and possible material loss.

Another problem encountered was sample breakthrough/crashing during FSPE caused by fluorous tags not sticking to the column. While 80/20 MeOH—H₂O is the standard solvent in the art for loading sample onto the column, the present inventors determined that product loading could be improved by substituting a less fluorophilic solvent for the standard solvent combination. Examples of preferred solvents for this purpose include N,N-dimethylformamide and acetonitrile.

Further, the inventors found an undesired amount of solvent evaporation occurred as a result of the heat generated by the machine. One means of reducing this solvent evaporation was to change the reagent stock solution to one having a higher boiling temperature. Another means of reducing the level of heat and, thus, reducing solvent evaporation, is to increase the level of insulation in the reactor vials. Other means of reducing heat in the system would be readily ascertainable by persons skilled in the art.

Moreover, the machine provided an unacceptable amount of sample splashing out of the FSPE cartridge during loading. The inventors remedied this by increasing the equilibrium time for the pressure in the FSPE to equilibrate.

In a preferred embodiment of the invention, the inventors employed special conical vials in order to minimize the amount of product that is eluted from the solid-phase extraction cartridges that could not be transferred by the flat-tip needle back to the reaction vials.

The reaction processes and solid-phase processes are preferably performed under an inert atmosphere to exclude water. The robot arm is programmed to remove a portion of the reaction mixture after each reaction and aliquot the mixture to a separate vial. This aliquot can be tested for reaction progress to stop the automated process when desired. When removed from the apparatus, oligosaccharides are purified by conventional means, then either used directly or reintroduced to the apparatus for further reaction cycles.

The process of the invention is surprising in that the described non-covalent fluorous-fluorous interactions were found by the inventors to be sufficiently robust to have a robot carry out the purification steps with reliable collection of the desired fluorous-tagged product, without human intervention, for subsequent reaction steps. The technology is also applicable to automated, iterative synthesis of other smaller-sized molecules (i.e. 8 octimers or less) such as, but not limited to, polyketides, glycosylated peptides, oligopeptides, and other molecules of eight octimer or smaller.

The following examples are offered to illustrate but not limit the invention. Thus, they are presented with the understanding that various formulation modifications as well as method of delivery modifications may be made and still be within the spirit of the invention.

### EXAMPLE 1

### Automated Synthesis of Polyrhamnose and Polymannose

An illustration of the foregoing procedure is set forth as FIG. 5.

### 1. Sample Preparation

Donor molecule (265.4 mg, 500 μmol) was dissolved in anhydrous 1,2-dichloroethane (DCE, 2.5 ml) in an 8-mL vial and placed on the stock solution rack (Donor 1) (FIG. 5) under nitrogen. Trimethylsilyltrifluoromethanesulfonate (TMSOTf) in DCE (0.27 M, 1.05 mL) prepared in a 13-mL vial was placed as indicated on the stock solution rack under nitrogen. Anhydrous toluene (20 mL) was placed in a 100-mL vial and clamped to the stock solution rack under nitrogen. Methanol (100 mL), 80% methanol/water (100 mL), and triethylamine (30 mL) were prepared in 200-mL stock solution bottles and connected to the transfer ports. Acceptor molecule (43.7 mg, 50 μmol) was dissolved in anhydrous DCE (0.5 ml) in an 8-mL conical vial (Wheaton E-Z extraction vial) capped with pre-punctured septa and placed at the reagent rack where indicated (F-tagged OH).

A sodium methoxide solution in methanol (0.5 M, 5 mL) was prepared in an 8-mL vial capped with a pre-punctured septa and placed at the reagent rack where indicated. An acetic acid solution in methanol (0.2 M, 5 mL) was prepared in an 8-mL vial capped with pre-punctured septa and placed at the reagent rack where indicated. N,N-dimethylformamide (DMF, 5 mL) was transferred to an 8-mL vial capped with pre-punctured septa and placed at the reagent rack where indicated. A fluorous solid phase extraction (FSPE) cartridge (2 g, 10 cc) was preconditioned with 80% methanol and placed in the machined SPE block. An empty 8-mL conical vial (Wheaton E-Z extraction vial) was placed under the FSPE cartridge. DCE (1 L) was placed as the reservoir solution for rinsing.

### 2. Cleaning Cycle

Before each run, the cleaning cycle method was employed. During the cycle, each individual reactor vial is rinsed with methanol (6 mL) and DCE (6 mL) 3 times each. When all the solvent is removed, the reactor vials are kept at 70° C. for 45 min under reduced pressure.

### 3. Method Run

### 3.1. Glycosylation

Reactor vials were cooled to 0° C. during the 5 min wait time by the heat transfer oil. Then, a flat-tipped needle transferred the acceptor molecule (F-tagged OH) solution (0.6 mL) to the reaction vial 1 (FIG. 5), followed by the transfer of the acceptor molecule solution (0.5 mL) and the TMSOTf solution (56 μl). After each individual transfer, the needle was rinsed by DCE (2 mL) inside and out before operating the next task. The reaction mixture was vortexed at 800 rpm for 30 min at 0° C. under inert gas. After the reaction time, the needle withdrew 30 μl from the reaction mixture and placed it into the first well of the microtiterplate for thin layer chromatography monitoring. Triethylamine (0.5 mL) was added to the solution for quenching and solvent evaporated under reduced pressure.

### 3.2. Deacetylation

To the resulting residue, methanol (0.5 mL) was added to the reactor vial followed by the sodium methoxide solution (0.4 mL). The reaction mixture was vortexed at 800 rpm for 45 min at ambient temperature. After the reaction time the needle withdrew 30 μl from the reaction mixture and placed it into the second well of the microtiterplate for thin layer chromatography monitoring. Acetic acid solution (0.75 mL) was added to the reactor vial for quenching followed by addition of toluene (1 mL); solvent was then removed under reduced pressure.

### 3.3. FSPE

DMF (0.4 mL) was added to the crude mixture and the vials were vortexed at 100 rpm for 5 min. The reaction mixture (0.7 mL) was transferred to the FSPE cartridge at the

SPE rack and dispensed at a speed of 20 mL/s via the 1 mL syringe. Then 80% methanol (4.5 mL) was used to rinse the empty reactor vial and the resulting solution was also delivered to the FSPE cartridge. Additional 80% methanol solution (1 mL) was used to rinse the FSPE cartridge. During the 80% methanol rinse, the cartridge was positioned at “SPE waste” for the eluted mixture to be disposed. Methanol (1.5 mL, repeated 3 times) was used to wash the FSPE cartridge for eluting the desired compound. During the task, the FSPE cartridge was positioned as “SPE collect” to be placed right above the 8-mL vial for collection of the sample. After the task, the position of the SPE rack was changed into “SPE direct” for the needle to withdraw the collected sample from the vial and deliver it to the clean reactor vial for the next reaction. Toluene (1 mL) was added to the solution and solvent was evaporated under reduced pressure. After the evaporation task, once again toluene (1 mL) was added and removed under reduced pressure to remove residual water.

3.5 cycles were completed for the synthesis of rhamnose pentasaccharide.

For the synthesis of a polymannose heptasaccharide, the same protocol was applied. FIG. 6 illustrates the specific procedures. The specific operation conditions are described in Table 1 below.

TABLE 1
Cycle descriptions used for the synthesis of mannose heptasaccharide

| | | | Operation |
|---|---|---|---|
| Step | Task | Reagents/Operation | time |
| 1 | Glycosylation | 2 equivalent donor (100 μmol), | 30 min |
| | | 0.3 equivalent TMSOTf | |
| 2 | TLC sample | 30 μL of crude reaction mixture | |
| | | withdrawn | |
| 3 | Quenching | 0.5 ml TEA | |
| 4 | Evaporation | 40° C. | 45 min |
| 5 | Deacetylation | 4 equivalent of NaOMe solution | 45 min |
| 6 | TLC sample | 30 μL of crude reaction mixture | |
| | | withdrawn | |
| 7 | Quenching | 0.7 mL Acetic acid solution | |
| 8 | Evaporation | 40° C. | 45 min |
| 9 | FSPE | 0.4 ml DMF | |
| | preparation | | |
| 10 | Sample | 0.7 mL crude sample transferred to | |
| | loading | cartridge | |
| 11 | Wash | 1.5 mL 80% methanol wash | |
| | | (repeated 3 times) | |
| 12 | Wash | 1.5 mL methanol wash | |
| | | (repeated 3 times) | |
| 13 | Transfer | 4.8 mL collected sample transferred | |
| | | to clean vial | |
| 14 | Evaporation | 40° C. | 45 min |
| 15 | Transfer | 1 mL toluene added | |
| 16 | Evaporation | 40° C. | 45 min |

6.5 cycles were completed for the synthesis of mannnose heptasaccharide.

Persons skilled in the art will readily appreciate that the processes described above may in some instances be combined or separated into several steps. Further, persons skilled in the art will also readily appreciate that the processes of this invention may be accomplished using a variety of equipment and techniques that are well known in the art. The specific equipment and processes used are not crucial so long as the intended result is accomplished.

It should be appreciated that minor modifications of the composition and the ranges expressed herein may be made and still come within the scope and spirit of the present invention.

Having described the invention with reference to particular compositions, theories of effectiveness, and the like, it will be apparent to those of skill in the art that it is not intended that the invention be limited by such illustrative embodiments or mechanisms, and that modifications can be made without departing from the scope or spirit of the invention, as defined by the appended claims. It is intended that all such obvious modifications and variations be included within the scope of the present invention as defined in the appended claims. The claims are meant to cover the claimed components and steps in any sequence which is effective to meet the objectives there intended, unless the context specifically indicates to the contrary.

What is claimed is:

1. An improved process for iterative fluorous solution-phase synthesis of biomolecules comprising the steps of:
tagging the protected molecule with a soluble fluorous tag;
deprotecting the protected molecule to produce a reactive end on the molecule; and
coupling a protected donor molecule to the reactive end of the fluorous-tagged molecule to form a coupled molecule; and
purifying the coupled molecule by separating the soluble fluorous-tagged compounds from the nonfluorous-tagged compounds, said process being automated.

2. The process of claim 1 whereby the biomolecule synthesized is an oligosaccharide, the molecule is a monomer, and the donor molecule is a glycosyl donor.

3. The process of claim 1 whereby the process is automated by a robotic driven automated workstation.

4. The process of claim 1 that is repeated at least once.

5. The process of claim 1 whereby the process requires 3 or fewer equivalents of donor molecule.

6. The process of claim 1 whereby the purification step comprises filtering the coupled molecule to remove impurities; said filtration occurring at greater than ambient pressure.

7. The process of claim 1 further including the step of removing any uncoupled donor molecule from the reaction by evaporation.

8. The process of claim 7 whereby the uncoupled donor molecule is in a reaction mixture, and the uncoupled donor molecule is removed by evaporation with a solvent that lowers the viscosity of the reaction mixture.

9. The process of claim 8 whereby the solvent is toluene or benzene.

10. The process of claim 1 whereby the FSPE step is accomplished by loading the coupled molecule onto a column whereby the fluorous tag sticks to the column, said coupled molecule being present in a fluorophobic solvent to form a product sample.

11. The process of claim 10 further providing the step of allowing pressure in the product sample to equilibrate prior to loading the product sample onto the column.

12. The process of claim 1 that is performed under an inert atmosphere.
